(19) United States
(12) Patent Application Publication
(10) Pub. No.: US 2007/0190520 A1
(43) Pub. Date: Aug. 16, 2007

(54) METHOD OF CHARACTERIZING A BIOLOGICALLY ACTIVE COMPOUND

(76) Inventors: David A. WOLF, Houston, TX (US); Donnie RUDD, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/669,637

(22) Filed: Jan. 31, 2007

Related U.S. Application Data
(60) Provisional application No. 60/764,524, filed on Feb. 2, 2006.

(51) Int. Cl. C12Q 1/70; C12Q 1/68; C12Q 1/00

(52) U.S. Cl. 435/4; 435/5; 435/6; 977/902

(57) ABSTRACT

A method of characterizing a biologically active compound by placing a cell mixture into a rotatable bioreactor to initiate a three-dimensional culture comprising a biological component and at least one cell, controllably expanding the cells in the rotatable bioreactor and testing the biological component to characterize the biologically active compound. The present invention may also preferably comprise exposing the cells to a time varying electromagnetic force.

### CROSS REFERENCE TO RELATED APPLICATIONS

The present application claims priority from U.S. Ser. No. 60/764524 filed Feb. 2, 2006, and titled “Process for Testing Drug Efficacy”.

### FIELD OF THE INVENTION

The present invention relates generally to the field of characterizing a biologically active compound. More specifically, the present invention relates to a method of controllably expanding a three-dimensional culture in a rotatable bioreactor to characterize a biologically active compound.

### BACKGROUND OF THE INVENTION

Most biologically active compounds target tissue specific functions that are based on the detailed structures and chemical processes occurring at all levels of biological processes from molecular through large-scale tissue structure. Testing such biologically active compounds for efficacy and determining the mechanism of action requires high fidelity cells and tissue and is usually conducted in conventional in-vitro culture (for gross effects), animals, and finally in human clinical trials. Each of these methods has limitations, however, introduced by either low fidelity and/or ethics. Furthermore, the ability to investigate the specific detailed mechanism or physical site of a biologically active compounds action is limited by these conventional methods of testing. A similar case is true for understanding the mechanism and degree of toxicity for toxic chemicals and materials or for understanding or characterizing the biological activity of a reagent. In the case of using animals for the testing, the biological environment is too complex, not controllable, rich in confounding factors, often poorly represents the human condition, and suffers ethical limits. Conventional cultures, such as two-dimensional cultures, or those that require agitation, stirring, and other ways of mixing the culture, are not able to reproduce biologically active interactions with cells as they would interact in the in vivo tissue microenvironment. Other culture techniques utilizing fixed matrices in conventional non-rotating systems, i.e. absent any component of freely suspended rotating material also introduce limitations on the fidelity, accuracy, analyzability, and practicality for conducting these studies. Human testing introduces obvious severe ethical constraints along with many of those inherent in animal testing.

Structural relationships of the primary functional cells to each other, to support cells, and to mechanical support substrate permit accurate and natural cell and tissue specific behavior. Features such as junctional complexes, gland formation, cell polarity, and overall correct geometrical relationships to support cells, and acellular components mediate such cell and tissue specific behavior. Moreover, individual cells and tissues function in a manner dependent on these, and other, features. Other features also contribute to the relationships between and among cells and the three-dimensional interactions between cells in the larger tissue structure including mucin, secreted hormones (insulin from pancreatic Beta cells), intercellular soluble signals, cell membrane surface markers, membrane bound enzymes, immune identity markers, adherence molecules, vacuoles, stored and released neurotransmitters, and cellular internal specialized machinery such as myosin contractile fibers in the case of muscle, glycogen and conjugational toxic clearance processing in the case of hepatocytes. Individual cell functions and cell-to-cell interactions are dependent on these and other features.

The efficacy and toxicity of biologically active compounds are tested and measured by determining the effect the biologically active compound has on the cell, tissue, and/or these features. Such measurable responses include genetic expression, karyotype, growth rate characteristics, multi-cellular and individual cellular morphology, metabolic measures, and inter-cellular relationships. These and other responses are well known but the difficulty has been that traditional culture methods are unable to grow a sufficient amount of cells and tissue so that cells and cellular interactions substantially mimic the in vivo situation and any responses to biologically active compounds would be an accurate reflection of the in vivo cellular response to the biologically active compound. Therefore, traditional culture systems, which do not support cellular and tissue vast and accelerated growth over extended periods of time, do not provide an accurate in vitro model for characterizing biologically active compounds by testing their effects.

Growth of a variety of both normal and neoplastic mammalian tissues in both mono-culture and co-culture has been established in both batch-fed and perfused rotating wall vessels, Schwarz et al., U.S. Pat. No. 4,988,623, (1991) and Schwarz et al., U.S. Pat. No, 5,026,590, (1991), and in conventional plate or flask based culture systems. In some applications, growth of three-dimensional structure, e.g., tissues, in these culture systems have been facilitated by support of a solid matrix in the form of biocompatible polymers and microcarrier. In the case of spheroidal growth, three-dimensional structure has been achieved without matrix support, Goodwin, et al., In Vitro Cell Dev. Biol., 28A: 47-60(1992), Goodwin, et al., Proc. Soc. Exp. Biol. Med., 202:181-192 (1993), Goodwin, et al., J. Cell Biochem., 51:301-311 (1993), Goodwin, et al., In Vitro Cell Dev. Biol. Anim., 33:366-374 (1997). However, human tissue has been largely refractory, in terms of controlled growth induction and three-dimensional organization, under conventional culture conditions. Actual microgravity, and to a lesser extent, rotationally simulated microgravity, have permitted enhanced cell growth.

Attempts have also been made to use static electric fields to enhance nerve growth in culture. Embryonic development has been successfully altered and isolated nerve axon directional growth has been successfully achieved. However, actual acceleration of potentiation of growth or genetic activity causing such, have not been achieved. Mechanical devices intended to help grow and orient three-dimensional mammalian neuronal tissue are currently available. Fukuda et al., U.S. Pat. No. 5,328,843 used zones formed between stainless steel shaving blades to orient neuronal cells or axons. Additionally, electrodes charged with electrical potential were employed to enhance axon response. Aebischer, U.S. Pat. No. 5,030,225, described an electrically charged, implantable tubular membrane for use in regenerating severed nerves within the human body. Wolf, et al., U.S. Pat. No. 6,485,963, utilized electromagnetic force to increase cell growth, but in many cases the cell growth, or expansion, did not occur rapidly enough for needed testing or treatment of a patient.

There remains a need, therefore, for an in vitro culture system that essentially mimics the in vivo microenvironment for testing a biologically active compounds' effects on cells and tissues, thus providing responses that are highly representative of the in vivo situation.

### SUMMARY OF THE INVENTION

The present invention relates to a method of characterizing a biologically active compound comprising placing a cell mixture into a rotatable bioreactor to initiate a three-dimensional culture wherein the three-dimensional culture comprises cells and a biological component, controllably expanding the cells in the three-dimensional culture while at the same time maintaining the cells three dimensional geometry and cell-to-cell support and geometry by rotating the rotatable bioreactor, introducing a biologically active compound into the three dimensional culture, and testing the biological component using a test to characterize the biologically active compound.

### BRIEF DESCRIPTION OF THE DRAWINGS

FIG. 1 is an elevated side view of a preferred embodiment of a rotatable bioreactor;

FIG. 2 is a side perspective of a preferred embodiment of the rotatable bioreactor;

FIG. 3 schematically illustrates a preferred embodiment of a culture carrier flow loop of a rotatable bioreactor;

FIG. 4 is the orbital path of a typical cell in a non-rotating reference frame;

FIG. 5 is a graph of the magnitude of deviation of a cell per revolution; and

FIG. 6 is a representative cell path as observed in a rotating reference frame of the culture medium.

### DETAILED DESCRIPTION OF THE PREFERRED EMBODIMENTS

In the simplest terms, a rotatable bioreactor comprises a culture chamber that, in operation, can be rotated about a substantially horizontal axis, and has an interior portion and an exterior portion. The interior portion of the culture chamber defines a space that may removably receive a biological component mixture. Preferably, the culture chamber is substantially cylindrical. In a preferred embodiment of the rotatable bioreactor, an electrically conductive coil is wrapped around the exterior portion of the culture chamber preferably affixed to the culture chamber, more preferably removably affixed to the culture chamber. A TVEMF source is operatively connected to the electrically conductive coil so that, in use, the TVEMF source delivers a TVEMF to the interior portion of the culture chamber and to the biological component mixture to expand the biological component therein. The culture chamber has at least one aperture so that, when in use, the biological component mixture may be placed into the interior portion of the culture chamber. The aperture may also preferably be used for the exchange of culture medium and/or a biologically active compound, and the removal of samples of the biological component for testing, and preferably the aperture is fitted for use with a syringe.

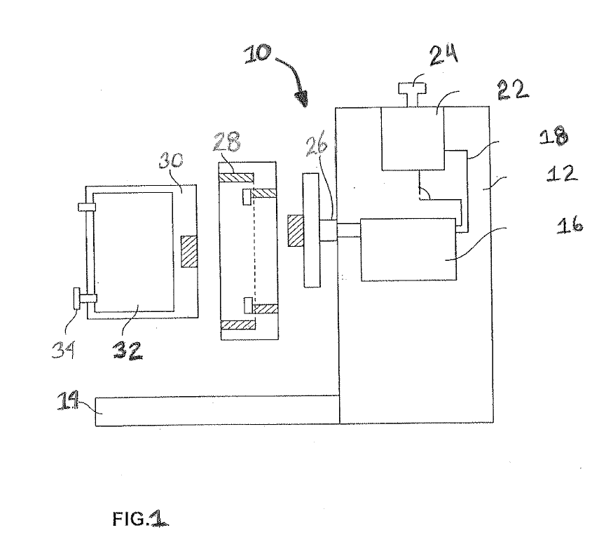

In the drawings, FIG. 1 is a cross sectional elevated side view of a preferred embodiment of a rotatable bioreactor 10. In this preferred embodiment a motor housing 12 is supported by a base 14. A motor 16 is affixed inside the motor housing 12 and connected by a first wire 18 and a second wire 20 to a control box 22 that houses a control device therein whereby the speed of the motor 16 can be incrementally controlled by turning the control knob 24. Extending from the motor housing 12 is a motor shaft 26. A rotatable mounting 28 removably receives a rotatable bioreactor holder 30 that removably receives a culture chamber 32 preferably disposable and also preferably substantially cylindrical, which is affixed, preferably removably, within the rotatable bioreactor holder 30, preferably by a screw 34. The culture chamber 32 is mounted, preferably removably, to the rotatable mounting 28. The rotatable mounting 28 is received by the motor shaft 26. In use, when the control knob 24 is turned on, the culture chamber 32 is rotated. By the term “rotated” and similar terms it is intended that, in use, the rotation of the culture chamber prevents collision of the cells, tissue, or cell mass, with the interior portion of the rotatable TVEMF bioreactor. The culture chamber may also preferably be perfused.

The culture chamber of the rotatable bioreactor 10 of the present invention may preferably be disposable meaning that it can be discarded and a new one used in later cultures as needed. The rotatable bioreactor 10 may also preferably be sterilized, for instance in an autoclave, after each use and re-used for later cultures. A disposable culture chamber 32 could be manufactured and packaged in a sterile environment thereby enabling it to be used by the medical or research professional much the same as other disposable medical devices are used.

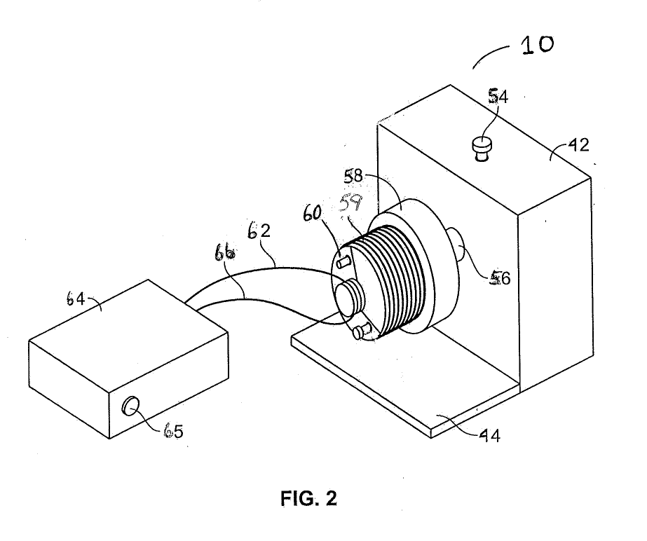

FIG. 2 is a side perspective of a rotatable bioreactor 10. FIG. 2 illustrates the motor housing 42 retaining a control knob 54 and supported by a base 44. Extending from the motor housing 42 is a motor shaft 56. A rotatable mounting 58 removably receives a rotatable bioreactor holder 60 that removably receives a culture chamber. An electrically conductive coil 59 is wrapped around the exterior portion of the culture chamber. The electrically conductive coil 59 may preferably be made of any electrically conductive material that conducts electricity including, but not limited to, the following conductive materials; silver, gold, copper, aluminum, iron, lead, titanium, uranium, a ferromagnetic metal, and zinc, or a combination thereof. The electrically conductive coil 59 may also preferably comprise salt water. The electrically conductive coil 59 1nay also preferably be a solenoid. Furthermore, the electrically conductive coil 59 may preferably be contained in an electric insulator comprising, but not limited to, rubber, plastic, silicones, glass, and ceramic. The electrically conductive coil 59 may be wrapped around the exterior portion of the culture chamber, and thereby, the culture chamber supports a shape of the electrically conductive coil 59, preferably having a substantially oval cross-section, more preferably a substantially elliptical cross-section, and most preferably a substantially circular cross-section. The electrically conductive coil 59 that is integral with a culture chamber that is preferably disposable is installed into the rotatable bioreactor 10 along with the disposable culture chamber and operatively connected to a TVEMF source 64. When the disposable culture chamber is discarded, the electrically conductive coil 59 is discarded therewith.

At a first end a first conductive wire 62 and a second conductive wire 66, both of which are integral with the electrically conductive coil 59, are operatively connected to a TVEMF source 64 having a source knob 65 which, in use, can be turned on to generate a TVEMF. At a second end the wires 62, 66 are connected to at least one ring to facilitate the rotation of the electrically conductive coil 59. When the control knob 54 is turned on, the culture chamber and the electrically conductive coil 59 are rotated simultaneously. Furthermore, the electrically conductive coil 59 remains affixed to, and encompassing, the culture chamber, so that in use, it supplies a TVEMF to the cells in the culture chamber.

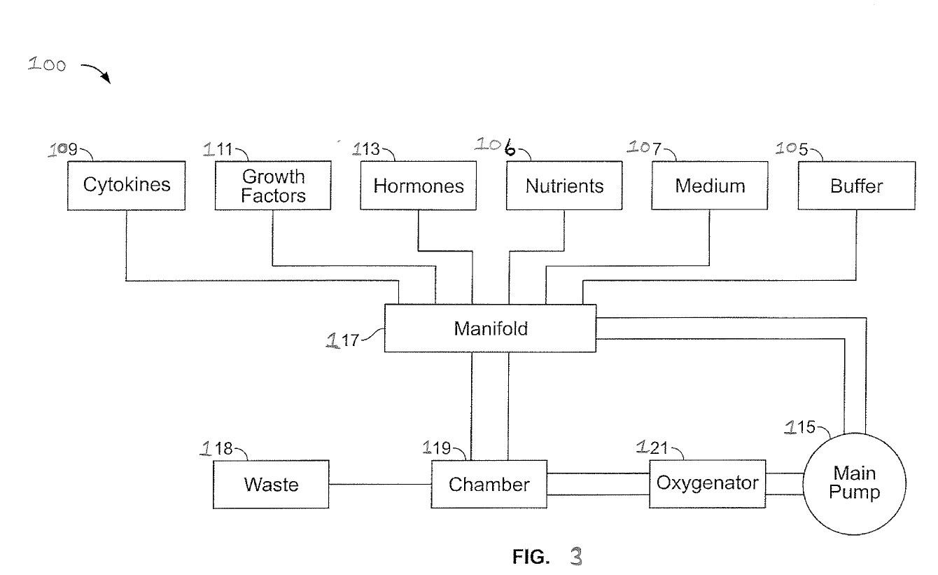

The culture chamber of a rotatable bioreactor may preferably be fitted with a culture medium flow loop 100 for the support of respiratory gas exchange in, supply of nutrients in, and removal of metabolic waste products from a three-dimensional culture. A preferred embodiment of a culture medium flow loop 100 is illustrated in FIG. 3, having a culture chamber 119, an oxygenator 121, an apparatus for facilitating the directional flow of the culture medium, preferably by the use of a main pump 115, and a supply manifold 117 for the selective input of culture medium requirements such as, but not limited to, nutrients 106, buffers 105, fresh medium 107, cytokines 109, growth factors 111, and hormones 113. In this preferred embodiment, the main pump 115 provides fresh culture medium from the supply manifold 117 to the oxygenator 121 where the culture medium is oxygenated and passed through the culture chamber 119. The waste in the spent culture medium from the culture chamber 119 is removed, preferably by the main pump 115, and delivered to the waste 118 and the remaining volume of culture medium not removed to the waste 118 is returned to the supply manifold 117 where it may preferably receive a fresh charge of culture medium requirements before recycling by the pump 115 through the oxygenator 121 to the culture chamber 119.

In this preferred embodiment of a culture medium flow loop 100, adjustments are made in response to chemical sensors (not shown) that maintain constant conditions within the culture chamber 119. Controlling carbon dioxide pressures and introducing acids or bases corrects pH. Oxygen, nitrogen, and carbon dioxide are dissolved in a gas exchange system (not shown) in order to support cell respiration. The culture medium flow loop 100 adds oxygen and removes carbon dioxide from a circulating gas capacitance. Although FIG. 3 is one preferred embodiment of a culture medium flow loop that may be used in the present invention, the invention is not intended to be so limited. The input of culture medium requirements such as, but not limited to, oxygen, nutrients, buffers, fresh medium, cytokines, growth factors, and hormones into a rotatable TVEMF bioreactor can also be performed manually, automatically, or by other control means, as can be the control and removal of waste and carbon dioxide.

As various changes could be made in rotatable TVEMF bioreactors such as are contemplated in the present invention, without departing from the scope of the invention, it is intended that all matter contained herein be interpreted as illustrative and not limiting.

### DETAILED DESCRIPTION OF THE INVENTION

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “TVEMF” refers to “time varying electromagnetic force”. As discussed above, the TVEMF of this invention is in a delta wave, more preferably a differential square wave, and most preferably a square wave (following a Fourier curve). The TVEMF is preferably selected from one of the following: (1) a TVEMF with a force amplitude less than 100 gauss and slew rate greater than 1000 gauss per second, (2) a TVEMF with a substantially low force amplitude bipolar square wave at a frequency less than 100 Hz., (3) a TVEMF with a substantially low force amplitude square wave with less than 100% duty cycle, (4) a TVEMF with slew rates greater than 1(000 gauss per second for duration pulses less than 1 ms., (5) a TVEMF with slew rate bipolar delta function-like pulses with a duty cycle less than 1%, (6) a TVEMF with a force amplitude less than 100 gauss peak-to-peak and slew rate bipolar delta function-like pulses and where the duty cycle is less than 1%, (7) a TVEMF applied using a solenoid coil to create uniform force strength throughout the three-dimensional culture, (8) and a TVEMF applied utilizing a flux concentrator to provide spatial gradients of magnetic flux and magnetic flux focusing within the three-dimensional culture. The range of frequency in oscillating electromagnetic force strength is a parameter that may be selected for achieving the desired stimulation of the cells in the three-dimensional culture. However, these parameters are not meant to be limiting to the TVEMF of the present invention, as such may vary based on other aspects of this invention. The TVEMF may be measured for instance by standard equipment such as an EN131 Cell Sensor Gauss Meter.

As used throughout this application, the term “electrically conductive coil,” refers to any electrically conductive material that conducts electricity including, but not limited to, the following conductive materials; silver, gold, copper, aluminum, iron, lead, titanium, uranium, a ferromagnetic metal, and zinc, or a combination thereof. The electrically conductive coil may also preferably comprise salt water. The electrically conductive coil may also preferably be a solenoid. Furthermore, the electrically conductive coil may preferably be contained in an electric insulator comprising, but not limited to, rubber, plastic, silicones, glass, and ceramic. The electrically conductive coil may be wrapped around the exterior portion of the culture chamber of the rotatable TVEMF bioreactor, and therefore, preferably the culture chamber supports a shape of the electrically conductive coil, preferably having a substantially oval cross-section, more preferably a substantially elliptical cross-section, and most preferably a substantially circular cross-section. The culture chamber supports a shape of the electrically conductive coil preferably because the shape of the culture chamber and the shape of the electrically conductive coil are substantially similar. By “wrapped around,” it is intended that the electrically conductive coil encompasses the culture chamber so that preferably, in operation, a substantially uniform TVEMF is delivered to the interior portion of the culture chamber and the cells therein. By “encompasses” it is meant that the electrically conductive coil surrounds the culture chamber, and in use, delivers a preferably substantially uniform TVEMF to the interior portion of the culture chamber.

As used throughout this application, the term “biological component” refers to a portion of the three-dimensional culture in the rotatable bioreactor during the controllable expansion step of the method of the present invention. The biological component may preferably be tested during the culture or by further means after the culture is complete or even killed for special analytical techniques, such as electron microscopy. The biological component is tested to characterize a biologically active compound. The biological component may preferably be cells in any form, for instance activated T-cells, and any part of the cell including the membrane, the cell wall (in the case of plants), and/or the internal cell organelles including the mitochondria. The biological component to be tested may also preferably include secreted material, for instance mucin, collagen, and matrix, secreted hormones (insulin from pancreatic Beta cells), secreted intercellular structural components, introduced structural matrices, adherence matrices, growth substrates, intercellular soluble signals, cell membrane surface markers, membrane bound enzymes, immune identity markers, adherence molecules, vacuoles, stored and released neurotransmitters, and cellular internal specialized machinery such as myosin contractile fibers in the case of muscle, glycogen, culture media, compounds under test, suspected toxins under test, reagents under test, fungus, and conjugated complexes in the case of hepatocytes. A biological component may also preferably be a virus that is contained in the three-dimensional culture in the rotatable bioreactor during expansion. Such viruses may include, but are not limited to, HIV, Bird Flu, SIV, Hepatitis, HPV, the Herpes Virus, which may contain viral DNA, or in the case of retroviruses, viral RNA and particles. The biological component may also preferably be bacterial cells. The biological component may also preferably be any other nucleases, DNA, RNA, protein, artificial bioactive particles such as nano-particles, and/or genes, but is not limited thereto. The biological component may preferably be contained in the cell mixture, or added to the three-dimensional culture, or placed into the rotatable bioreactor before the addition of the cell mixture. The biological component is the focus of a test to characterize a biologically active compound.

As used throughout this application, the term “biologically active compound” refers to any biological substance, synthetic or non-synthetic, which is to be characterized by the method of the present invention. The biologically active compound may preferably be in any form including, but not limited to, powder, liquid, vapor, and gas. The biologically active compound can also preferably be, but is not limited to, protein, cells, chemicals, gasses, metals, growth factors, radiation, nano-particles, viruses, bacteria, and/or water, and/or any combinations thereof. The biologically active compound may also preferably be any material toxic to any portion of a three-dimensional culture, which comprises cells and a biological component. As used in this application the term toxin refers to any material or physical process, which is suspected or known to negatively affect a cell or tissues function or make it deviate from normal function. Toxins may preferably be heavy metals, and also preferably thermal, radiation, or even electrical exposures. Moreover, a biologically active compound may also preferably be a reagent that has an affect on a three-dimensional culture, which comprises cells and a biological component. As used throughout this application the term “reagent” refers to any material or physical process that is utilized to cause a change in cell or tissue function, architecture, structure, growth, lifespan, genetic composition, growth characteristics, secreted material, differentiation state, differentiation lineage predisposition, or surface marker expression, metabolic state, internal cell organelle structure, membrane structure, or tumorogenicity. In addition to the preferable biologically active compound such as insulin, transporting into a cell and causing glucose internal transport, a biologically active compound may preferably refer to reagent steps such as electroporation, chemoporation, and nanopartical interactions. The poration methods are particularly useful for production of hybridomas for monoclonal antibody production. Not to be bound by theory, but the well distributed three-dimensional culture contents enabled by rotating wall cultures are ideal for maximizing genetic exchange between the transformed and immunologic cells in the hybrid formation. This may improve the successful yield of desired hybrids producing desirable antibodies. Some additional preferred examples of a biologically active compound include, but are not limited to, insulin, interleukins, growth factors, differentiation modulators, chemotactic agents, inhibitors. According to the present invention, a biologically active compound can be characterized by testing its effects on a biological component.

As used throughout this application, the term “cells” refers to a cell in any form, for example, individual cells, tissue, cell aggregates, hybrid cells, cells pre-attached to cell attachment substrates for instance microcarrier beads, tissue-like structures, or intact tissue resections. The cells in this invention may also preferably be eukaryotic, more preferably prokaryotic. The cells that can be used in this invention are preferably mammalian, more preferably human, even more preferably adult stem cells, most preferably peripheral blood adult stem cells, and even more preferably mesenchymal cells. Other mammalian cells that can be used in the method of the present invention preferably include, but are not limited to, heart, liver, hematopoietic, skin, muscle, intestinal, pancreatic, central nervous system, cartilage, connective pulmonary, spleen, bone, and kidney.

As used throughout this application, the term “rotatable bioreactor” is meant to comprise a motor connected to a culture chamber with an interior portion and an exterior portion and which can be rotated at a speed. Preferably, the rotatable bioreactor is substantially cylindrical. The rotatable bioreactor may also preferably have an electrically conductive coil wrapped around the exterior portion of the culture chamber. Furthermore, the rotatable bioreactor may also have a culture medium flow loop affixed thereto to help facilitate the flow of culture medium to and through the three-dimensional culture therein. The flow of the culture medium through the culture chamber may be by perfusion. A TVEMF source may preferably be operatively connected to the electrically conductive coil. In use, a rotatable bioreactor may be rotated and, without being bound by theory, the rotation should be controlled to foster, support, and maintain a three-dimensional culture, as described for instance in the Description of the Invention. In a preferred embodiment having an electrically conductive coil, a TVEMF may be generated by the TVEMF source, and an appropriate gauss level, may preferably be delivered to the interior portion of the culture chamber via the electrically conductive coil. The volume of the rotatable bioreactor is preferably of from about 15 ml to about 2 L. See for instance FIGS. 1 and 2 herein for examples (not meant to be limiting) of a rotatable bioreactor.

The culture chamber of a rotatable bioreactor has rotatable culture chamber walls in the interior portion so that, in operation, the chamber walls are set into motion relative to the culture medium, and therefore, the three-dimensional culture, so that there is essentially no fluid stress sheer in the culture medium. The culture chamber also has at least one aperture for the addition and/or removal of culture medium, cells, and/or the biological component or portions thereof, and also for introducing a biologically active compound. The culture chamber of the rotatable bioreactor is substantially horizontally disposed. The culture chamber is also preferably substantially cylindrical with two ends, and is capable of rotation about a substantially horizontal axis. The culture chamber is preferably transparent in part so that the biological component, culture medium, and/or the three-dimensional culture therein can be assessed as needed. Furthermore, the culture chamber may also preferably be fitted with a microscope to assess the biological component, three-dimensional culture, and/or cells. Without being bound by theory, rotating the cells in a rotatable bioreactor provides for the controllable expansion of the cells over time, while at the same time, fostering, supporting, and maintaining the intricate three-dimensional geometry, cell-to-cell support and geometry of the cells.

As used throughout this application, the term “cell mixture” and similar terms, refers to a mixture of cells, preferably with another substance including, but not limited to, culture medium (with and without additives), plasma, buffer, and preservatives. The cell mixture may also comprise the biological component.

As used throughout this application, the term “three-dimensional culture,” refers to the cells and the biological component in the culture chamber of the rotatable bioreactor being controllably expanded by the method of the present invention. The cells in the three-dimensional culture have a three-dimensional geometry and cell-to-cell support and geometry fostered, supported, and maintained in the culture chamber. The cells in the three-dimensional culture have essentially the same three-dimensional geometry and cell-to-cell support and geometry as the cells in vivo. Three-dimensional tissue, non-necrotic cell mass, and/or tissue-like structures can also develop from the cells and be sustained and further expanded in the three-dimensional culture and at the same time mimic the in vivo microenvironment. The three-dimensional culture may be expanded (grown in number), sustained, or degenerated depending on the purpose of the experiment. In other words, depending on the effects of the biologically active compound and/or the preferred microenvironment needed to characterize the biologically active compound, the three-dimensional culture will be controllably expanded which could preferably mean expanding, maintaining, or degenerating the three-dimensional culture, or portions thereof.

As used throughout this application, the term “operatively connected,” and similar terms, is intended to mean that the TVEMF source can be connected, preferably removably, to the culture chamber in a manner such that, in operation, the TVEMF source imparts a TVEMF to the interior portion of the culture chamber of a rotatable bioreactor and the three-dimensional culture contained therein. The TVEMF source is operatively connected if, in use, it can impart a TVEMF to the interior portion of the culture chamber, preferably substantially uniform.

As used throughout this application, the term “exposing,” and similar terms, refers to the process of supplying a TVEMF to the three-dimensional culture contained in the interior portion of the culture chamber of a rotatable bioreactor. In operation, a TVEMF source is turned on and set at a preferred gauss range and a preferred waveform so that the same is delivered via the TVEMF source to an electrically conductive coil, wrapped around the exterior portion of the culture chamber of the rotatable bioreactor. The TVEMF is then delivered to the three-dimensional culture containing cells in the culture chamber thus exposing the cells to the TVEMF, preferably a substantially uniform TVEMF.

As used throughout this application, the term “culture medium” and similar terms, refers to a liquid comprising, but not limited to, growth medium and nutrients, which is meant for the sustenance of cells over time. The culture medium may be enriched with any of the following, but is not limited thereto; growth medium, buffers, growth factors, hormones, and cytokines. The culture medium is supplied to the cell mixture for suspension within the culture chamber of the rotatable bioreactor and to support expansion. The culture medium may preferably be mixed with the cell mixture before being added to the culture chamber of the rotatable bioreactor, or may more preferably be added to the culture chamber before the cell mixture is added thereby mixing the culture medium and cells in the rotatable bioreactor. The culture medium may preferably be enriched and/or refreshed during expansion as needed. Waste contained in the culture medium, as well as culture medium itself, may preferably be removed from the three-dimensional culture in the culture chamber during expansion as needed. Waste contained in the culture medium can be, but is not limited to, metabolic waste, dead cells, and other toxic debris. The culture medium can preferably be enriched with oxygen and preferably has oxygen, carbon dioxide, and nitrogen carrying capabilities.

As used throughout this applications, the term, “placing,” and similar terms, refers to the process of mixing the cell mixture and the culture medium before adding the cells to the rotatable bioreactor. The term “placing,” may also preferably refer to adding the cell mixture to culture medium that is already present in the rotatable bioreactor. The cells may preferably be placed into the rotatable bioreactor along with cell attachment substrates such as microcarrier beads.

As used throughout this application, the term “controllably expanding,” and similar terms, refers to the process of increasing, maintaining, or reducing the number of cells in a rotatable bioreactor by rotating the culture chamber. In a preferred embodiment, controllably expanding cells also comprises, exposing the three-dimensional culture to a TVEMF. Preferably, the cells are expanded without differentiation. If an increase in the number of cells is preferred, then the increase in number of cells per volume is expressly not due to a simple reduction in volume of fluid, for instance, reducing the volume of culture medium from 70 ml to 10 ml and thereby increasing the number of cells per ml. Controllably expanding cells by preferably expanding (increase in number) cells in a rotatable bioreactor provides for cells that have substantially the same three-dimensional geometry as the cells prior to expansion, preferably substantially the same geometry and cell to cell interactions as the cells display in the natural setting or tissue where they naturally exist, the in vivo microenvironment. Also preferably, controllably expanding may refer to sustaining a three-dimensional culture wherein, for instance, a preferred biologically active compound's effect is to prevent the number of cells to increase. More preferably, the three-dimensional culture may also be sustained to characterize the biologically active compound. Controllably expanding the cells in a three-dimensional culture of a rotatable bioreactor may also preferably refer to a degenerative culture wherein, for instance, a preferred biologically active compound's effect is to degenerate the three-dimensional culture. More preferably, the three-dimensional culture may intentionally be degenerated to characterize a preferred biological compound. Other aspects of expansion may also provide the exceptional characteristics of the cells of the present invention.

Preferably, cells and/or tissue undergo expansion for as long as is necessary to test a biological component to characterize a biologically active compound. The three dimensional culture may preferably undergo expansion for at least 160 days in a rotatable bioreactor.

As used throughout this application, the term “rotating,” and similar terms, refers to the rotation of the culture chamber of the rotatable bioreactor, which is preferably substantially cylindrical and is rotated about a substantially horizontal plane. Preferably, the rates of rotation range from about 1 revolutions per minute (RPM) to about 120 RPM, and more preferably from about 2 RPM to about 30 RPM. The rotatable bioreactor can preferably be automatically rotated, or manually rotated. In addition, the rate of rotation can preferably be manually adjusted, started, or stopped, or more preferably automatically adjusted, started, or stopped by using a sensor.

As used throughout this application, the term “introducing a biologically active compound” refers to the process of adding a biologically active compound into the culture chamber before, during, and/or after the step of controllably expanding. The biologically active compound may preferably be added as needed during the method of tile present invention and before and/or after various steps. Depending on the preferred test being performed, the biologically active compound can be added in different concentrations and at various times throughout the method of the present invention. The biologically active compound may also be inherently contained in the three-dimensional culture.

As used throughout this application, the term “testing” refers to the process of characterizing a biologically active compound by analyzing the biologically active compound's effect or non-effect on a biological component. Depending on the biological component to be tested, the tests will vary. For instance, if the biologically active compound is expected to effect the DNA or RNA of a biological component then the biologically active compound can be characterized by testing the effect on DNA by the polymerase chain reaction or RNA by the reverse transcriptase polymerase chain reaction. Other tests and methods of testing include, but are not limited to, the following instruments and techniques including: Mass Spectroscopy, flow cytometry, immunoflourescence, chromatography, mono- and bi-clonal antibodies, viability testing, toxicity tests, species tests, bioassays, dilution and effective concentration tests, dose response tests, hazardous waste tests, lethal concentration tests, screening tests, static renewal tests, cell number and tissue growth tests, and radiolabelling. Preferably, the present invention provides a method to characterize a biologically active compound by testing its effect on tumorogenicity and genetic abnormalities. Other examples of tests that can be performed by the method of the present invention to characterize a biologically active compound preferably include, but are not limited to, tests related to genetic expression, karyotype, growth rate characteristics, multi-cellular and individual cellular morphology, metabolic measures, and inter-cellular relationships. Tests would preferably be directed to measuring junctional complexes, gland formation, cell polarity, and geometrical relationships between cells (cell-to-cell geometry), and acellular components. The present invention provides a method comprising the step of controllably expanding cells so that the three-dimensional geometry of the cells remains as it is in the natural setting thereby providing a biological component for characterizing a biologically active compound by testing its effects of a biologically active compound on a biological component in a microenvironment that is essentially the same as is found in the in vivo situation. The biological component can be tested to characterize the biologically active compound's mechanisms of action, biological effects, efficacy, delivery, utility, and/or toxicity.

As used throughout this application, the term “characterize” refers to the process of determining the effect that a preferred biologically active compound has on a biological component by performing tests on the biological component. Tests can be performed preferably testing the efficacy and toxicity of the biologically active compound. The biological component can be tested to characterize the biologically active compound's mechanisms of action, biological effects, efficacy, delivery, utility, and/or toxicity.

As used throughout this application, the term “cell-to-cell geometry” refers to the geometry of cells including the spacing, distance between, and physical relationship of the cells relative to one another. For instance, in a preferred embodiment of the present invention, when utilizing cells, expanded cells, including those of tissues, cell aggregates, and tissue-like structures, the cells stay in relation to each other as in the in vivo microenvironment. The expanded cells are within the bounds of natural spacing between cells, in contrast to for instance two-dimensional expansion chambers, where such spacing is not preserved over time and expansion.

As used throughout this application, the term “cell-to-cell support” refers to the support one cell provides to an adjacent cell. For instance, tissues, cell aggregates, tissue-like structures, and cells maintain interactions such as chemical, hormonal, neural (where applicable/appropriate) with other cells. In addition, cells provide structural support for each other. It is not necessary for cells to be physically touching to provide cell-to-cell support. In the present invention, these interactions are maintained within normal functioning parameters, meaning they do not for instance begin to send toxic or damaging signals to other cells (unless such would be done in the natural cellular and tissue environment).

As used throughout this application, the term “three-dimensional geometry” refers to the geometry of cells in a three-dimensional state (same as or very similar to their natural state), as opposed to two-dimensional geometry for instance as found in cells grown in a Petri dish, where the cells become flattened and/or stretched. Not to be bound by theory, but the three-dimensional geometry of the cells is maintained, supported, and preserved such that the cell can develop into three-dimensional cell aggregates, tissues and/or tissue-like structures in the three-dimensional culture of the rotatable bioreactor, while at the same time, maintaining the three-dimensional geometry, and cell-to-cell support and geometry. By rotating the three-dimensional culture in the culture chamber, the cells therein can maintain a three dimensional geometry, cell-to-cell geometry and support, unlike cells grown in agitated environments such as shaking, using bubbles, and stirring. In addition, rotating the rotatable bioreactor keeps the cells in close proximity with one another so that they can establish and maintain the three-dimensional that is found in the cells in vivo microenvironment.

For each of the above three definitions, relating to maintenance of “cell-to-cell support” and “cell-to-cell geometry” and “three-dimensional geometry” of the cells of the present invention, the term “essentially the same” and “substantially the same,” means that natural geometry and support are provided in expansion, so that the cells not changed in such a way as to be for instance dysfunctional, toxic or harmful to the three-dimensional culture. Rather, the cells of the present invention, during and after expansion, mimic the in vivo situation.

In operation, a cell mixture is placed into the culture chamber of the rotatable bioreactor. In one preferred embodiment, the culture chamber is rotated over a period of time, while at the same time a TVEMF is generated in the culture chamber by the TVEMF source. By “while at the same time,” it is intended that the initiation of the delivery of the TVEMF may be before, concurrent with, or after rotation of the culture chamber is initiated. In a more complex rotatable bioreactor, a culture medium enriched with culture medium requirements preferably including, but not limited to, growth medium, buffer, nutrients, hormones, cytokines, and growth factors, which provides sustenance to the cells, can be periodically refreshed and removed. The biological component contained in the three-dimensional culture of the rotatable bioreactor can be tested at any time throughout the expansion process. Moreover, the biologically active compound being characterized can be introduced to the three-dimensional culture at any time before, during, or after the initiation of the three-dimensional culture in the rotatable bioreactor. By testing the biological component, the biologically active compound can be characterized.

In use, a rotatable bioreactor provides a stabilized culture environment into which cells may be introduced, suspended, assembled, grown, and maintained with improved retention or development of delicate three-dimensional structural integrity by simultaneously minimizing the fluid shear stress, providing three-dimensional freedom for cell and substrate spatial orientation, and increasing localization of cells in a particular spatial region for the duration of the expansion. In a preferred embodiment of controllably expanding cells in a rotatable bioreactor is provided these three criteria (hereinafter referred to as “the three criteria above”), and at the same time, the cells are exposed to a TVEMF. Of particular interest to the present invention is the dimension of the culture chamber, the sedimentation rate of the cells, the rotation rate, the external gravitational field, the TVEMF, and the biologically active compound and biological component interaction.

The present invention provides that even a cell degradative process in response to a biologically active compound well represent the degradative process in-vivo. For instance, characterizing a biologically active compound, such as a chemotherapeutic agent, by determining whether there is any reduction in the size and number of tumorogenic cells and tissue, and determining the mechanisms of action may involve testing a biological component associated with. Any successful tumor reduction in response to a chemotherapeutic agent would characterize the efficacy of the chemotherapeutic agent. In this case, the delivery of the biologically active compound into the tumor may be analyzed for penetration into or around cells and methods by which such delivery may be enhanced by other manipulations or drugs. Identifying the drug distribution in the culture tissue construct, within the internal cell sub-volumes, or on the cell surface is critical for precise understanding of efficacious drug delivery (toxic compound analyses, or reagent actions). The cultured model tumor is then analyzed for response to potential treatments (chemotherapeutic, radiation regimen, nanoparticle function, or combinations thereof) by conventional tissue and cell ultrastructural, molecular, immunologic, and physical analysis. In this preferred embodiment the biologically active compound consists of immuno-active elements such as antibody containing compounds or even living immune cells (which can be themselves modified such as by adoptive immunotherapeutic means—killer T-cell activation). As such, the biologically active compound may preferably contain a living cellular component which may be particularly well tested by the present invention given the freedom of movement for these elements so they may interact freely with the target tissue (tumor in this case).

The stabilized culture environment referred to in the operation of rotatable biroeactor is that condition in the culture medium, particularly the fluid velocity gradients, prior to introduction of cells, which will support a nearly uniform suspension of cells upon their introduction thereby initiating a three-dimensional culture upon addition of the cell mixture. In a preferred embodiment, the culture medium is initially stabilized into a near solid body horizontal rotation about an axis within the confines of a similarly rotating chamber wall of a rotatable bioreactor. In this condition the culture chamber walls are moving at the same angular rate as the c u l t u r e chamber contents because the start-up transients, and associated transient fluid velocity gradients, are dissipated. The culture chamber walls are set in motion relative to the culture medium so as to initially introduce rotation to the culture chamber contents. During this transient, which also occurs during culture chamber spin-down, significant fluid velocity gradients and associated fluid shear stresses, are present. After the culture chamber and contents reach steady state these gradients are significantly reduced and the fluid stress shear field therein is at a minimum. Cells are introduced to, and move through, the culture medium in the stabilized c u l t u r e environment thus initiating and maintaining a three-dimensional culture. The three-dimensional culture moves under the influence of gravity, centrifugal, and coriolus forces, and the presence of cells, particles, or any other elements, within the culture medium of the three-dimensional culture induces secondary effects to the culture medium. By the term “elements” it is meant to include anything present in the culture medium of the three-dimensional culture including, but not limited to, viruses, nano-particles, waste, dead cells, cells and any other objects therein. The significant motion of the culture medium with respect to the culture chamber, significant fluid shear stress, and other fluid motions, is due to the presence of these cells, particles, and/or elements within the culture medium.

It is also preferred that some of these elements may be fixed with the culture chamber wall rotation for convenience or advantage, with other elements free to move within the liquid compartment within the culture chamber. Such “fixed” elements may be objects (such as substrates) which would be otherwise too heavy to suspend by the rotating fluid alone, elements which are damaged by even the low sedimentation induced residual fluid shears within the culture chamber, adversely affected by inevitable wall impacts experienced by the freely suspended elements, for closer observation, or simply for operator convenience (such as to locate a particular element later. It is notable that introduction of such “fixed” elements represents an improvement in the culture process itself, independent of the biologically active compound tests which are the main subject of this document. For instance, an example would be to “hang” a heart valve shaped substrate within a rotating culture chamber as further cells are introduced for attachment onto that substrate in order to build an improved heart valve.

In most cases the cells with which the stabilized culture environment is primed sediment at a slow rate preferably under 0.5 centimeter per second. It is therefore possible, at this early stage of the three-dimensional culture, to select from a broad range of rotational rates (preferably of from about 1 to about 120 RPM, more preferably from about 2 to about 30 RPM) and chamber diameters (preferably of from about 0.5 to about 36 inches). Preferably, the slowest rotational rate is advantageous because it minimizes equipment wear and other logistics associated with handling of the three-dimensional culture.

Not to be bound by theory, rotation about a substantially horizontal axis with respect to the external gravity vector at an angular rate optimizes the orbital path of cells suspended within the three-dimensional culture. In operation, the cells expand to form a mass of cell aggregates, three-dimensional tissues, non-necrotic cell masses, and/or tissue-like structures, which increase in size as the three-dimensional culture progresses. The interactions between the cells such as the three-dimensional geometry and the cell-to-cell geometry and support essentially substantially mimics that found in the cells natural setting, the in vivo microenvironment. The progress of the three-dimensional culture is preferably assessed by a visual, manual, or automatic determination of an increase in the diameter of the three-dimensional cell mass in the three-dimensional culture. An increase or decrease in the size and/or number of the cell aggregate, tissue, non-necrotic cell mass, or tissue-like structure in the three-dimensional culture may require appropriate adjustment of the rotation speed in order to optimize the particular paths. The rotation of the culture chamber optimally controls collision frequencies, collision intensities, and localization of the cells in relation to other cells and also the limiting boundaries of the culture chamber of the rotatable TVEMF bioreactor. In order to control the rotation, if the cells are observed to excessively distort inwards on the downward side and outwards on the upwards side then the revolutions per minute (“RPM”) may preferably be increased. If the cells are observed to centrifugate excessively to the outer walls then the RPM may preferably be reduced. Not to be bound by theory, as the operating limits are reached, in terms of high cell sedimentation rates or high gravity strengths, the operator may be unable to satisfy both of these conditions and may be forced to accept degradation in performance as measured against the three criteria above.

The cell sedimentation rate and the external gravitations field place a lower limit on the fluid shear stress obtainable, even within the operating range of the rotatable bioreactor, due to gravitationally induced drift of the cells and/or elements through the culture medium of the three-dimensional culture. Calculations and measurements place this minimum fluid shear stress very nearly to that resulting from the cells' and/or elements' terminal sedimentation velocity (through the culture medium) for the external gravity field strength. Centrifugal and coriolis induced motion [classical angular kinematics provide the following equation relating the Coriolis force to an object's mass (m), its velocity in a rotating frame (vᵣ) and the angular velocity of the rotating frame of reference (□): F_{Coriolis}=−2 m (w x vᵣ)] along with secondary effects due to cell and culture medium interactions, act to further degrade the fluid shear stress level as the cells expand.

Not to be bound by theory, but as the external gravity field is reduced, much denser and larger three-dimensional structures can be obtained. In order to obtain the minimal fluid shear stress level it is preferable that the culture chamber be rotated at substantially the same rate as the culture medium. Not to be bound by theory, but this minimizes the fluid velocity gradient induced upon the three-dimensional culture. It is advantageous to control the rate and size of tissue formation in order to maintain the cell size (and associated sedimentation rate) within a range for which the rate of expansion is able to satisfy the three criteria above. However, preferably, the velocity gradient and resulting fluid shear stress may be intentionally introduced and controlled for specific research purposes such as studying the effects of shear stress on the three-dimensional cell aggregates. In addition, transient disruptions of the expansion process are permitted and tolerated for, among other reasons, logistical purposes during initial system priming, sample acquisition, system maintenance, and three-dimensional culture termination.

Rotating cells about an axis substantially perpendicular to gravity can produce a variety of sedimentation rates, all of which according to the present invention remain spatially localized in distinct regions for extended periods of time ranging from seconds (when sedimentation characteristics are large) to hours (when sedimentation differences are small). Not to be bound by theory, but this allows these cells sufficient time to interact as necessary to form multi-cellular structures and to associate with each other in a three-dimensional culture. The cells may preferably expand in the rotatable bioreactor as needed. The cells may preferably continue to expand in the rotatable bioreactor for at least 160 days.

Culture chamber dimensions also influence the path of cells in the three-dimensional culture of the present invention. A culture chamber diameter is preferably chosen which has the appropriate volume, preferably of from about 15 ml to about 2 L for the intended three-dimensional culture and which will allow a sufficient seeding density of cells. Not to be bound by theory, but the outward cells drift due to centrifugal force is exaggerated at higher culture chamber radii and for rapidly sedimenting cells. Thus, it is preferable to limit the maximum radius of the culture chamber as a function of the sedimentation properties of the tissues anticipated in the final three-dimensional culture stages (when the largest cell aggregates with high rates of sedimentation have formed).

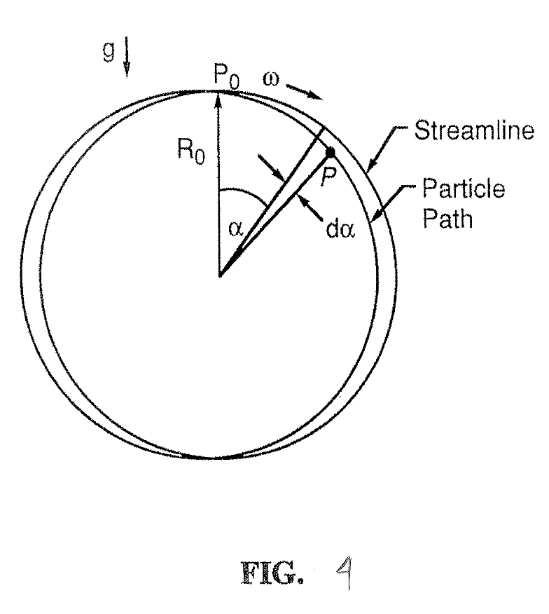

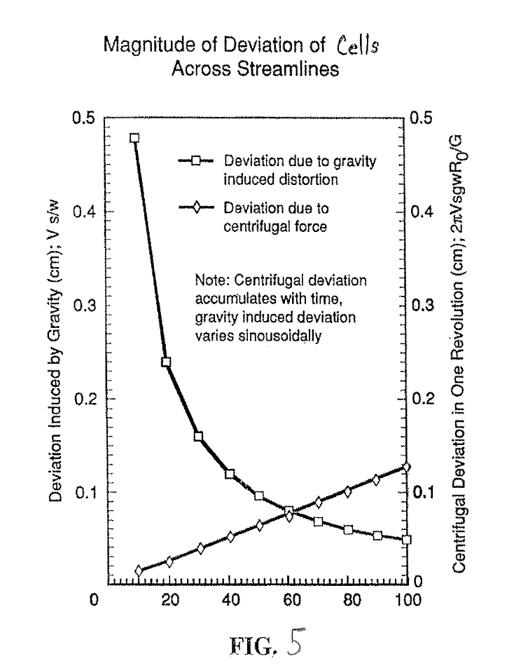

The path of the cells in the three-dimensional culture has been analytically calculated incorporating the cell motion resulting from gravity, centrifugation, and coriolus effects. A computer simulation of these governing equations allows the operator to model the process and select parameters acceptable (or optimal) for the particular planned three-dimensional culture. FIG. 4 shows the typical shape of the cell orbit as observed from the external (non-rotating) reference frame. FIG. 5 is a graph of the radial deviation of a cell from the ideal circular streamline plotted as a function of RPM (for a typical cell sedimenting at 0.5 cm per second terminal velocity). This graph (FIG. 5) shows the decreasing amplitude of the sinusoidally varying radial cells deviation as induced by gravitational sedimentation. FIG. 5 also shows increasing radial cells deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the expansion of cells of a given sedimentation rate in a given external gravity field strength. Not to be bound by theory, but as a typical three-dimensional culture is expanded the tissues, cell aggregates, and tissue-like structures increase in size and sedimentation rate, and therefore, the rotation rate may preferably be adjusted to optimize the same.

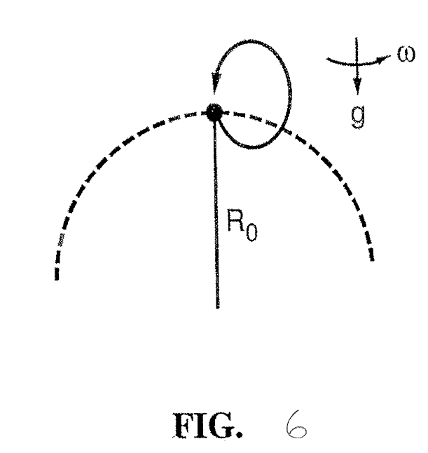

In the three-dimensional culture, the cell orbit (FIG. 4) from the rotating reference frame of the culture medium is seen to move in a nearly circular path under the influence of the rotating gravity vector (FIG. 6). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the rotatable bioreactor provides cells of differing sedimentation properties with sufficient time to interact mechanically and through soluble chemical signals thus mimicking substantially the same cell-to-cell support and geometry as is found in vivo. In operation, the present invention provides for sedimentation rates of preferably from about 0 cm/second up to 10 cm/second.

Furthermore, in operation the culture chamber of the present invention has at least one aperture preferably for the input of fresh culture medium, a cell mixture, a biological component, and a biologically active compound, and also the removal of a volumn of spent culture medium containing metabolic waste and samples of biological component, but not limited thereto. Preferably, the exchange of culture medium can also be via a culture medium loop wherein fresh or recycled culture medium may be moved within the culture chamber preferably at a rate sufficient to support metabolic gas exchange, nutrient delivery, and metabolic waste product removal. This may slightly degrade the otherwise quiescent three-dimensional culture. It is preferable, therefore, to introduce a mechanism for the support of preferred components including, but not limited to, respiratory gas exchange, nutrient delivery, growth factor delivery to the culture medium of the three-dimensional culture, and also a mechanism for metabolic waste product removal in order to provide a long term three-dimensional culture able to support significant metabolic loads for periods of hours to months.

The present invention preferably exposes the three-dimensional culture, and therefore the biological component and cells, to a TVEMF that not only provides for accelerated expansion of cells that maintain their three-dimensional geometry and cell-to-cell support and geometry, but in addition, may affect some properties of cells during expansion, for instance up-regulation of genes promoting growth, or down regulation of genes preventing growth. The electromagnetic field is generated by a TVEMF source. In operation, an electrically conductive coil of a rotatable bioreactor is preferably rotatable with the culture chamber, meaning about the same axis as the culture chamber and in the same direction. Also, the electrically conductive coil may preferably be fixed in relation to a culture chamber of a rotatable perfused TVEMF-bioreactor. The electrically conductive coil nay preferably be integral with, meaning affixed to and wrapped around the exterior portion of the culture chamber of the electrically conductive coil of the culture chamber of the rotatable TVEMF bioreactor. The TVEMF source is operatively connected to the rotatable TVEMF bioreactor. The method of the present invention provides these three criteria above in a manner heretofore not obtained and optimizes a three-dimensional culture, and at the same time, facilitates and supports expansion such that a sufficient expansion (increase in number per volume, diameter in reference to tissue, or concentration) is detected in a sufficient amount of time.

In addition to the qualitatively unique cells that are produced by the operation of the rotatable bioreactor, not to be bound by theory, an increased efficiency with respect to utilization of the total culture chamber volume for cell and tissue culture may be obtained due to the substantially uniform homogeneous suspension achieved. Advantageously, therefore, a rotatable bioreactor, in operation, provides an increased number of cells in the same rotatable bioreactor with less human resources. Many cell types may be utilized in this method. Fundamental cell and tissue biology research as well as clinical applications requiring accurate in vitro models of in vivo cell behavior are applications for which the present invention and method of using the same provides an enhancement because, as indicated above and throughout this application, the expanded cells and tissue of the present invention have essentially the same three-dimensional geometry and cell-to-cell support and cell-to-cell geometry as naturally-occurring, non-expanded cells and tissue. Testing a biologically active compound in an environment that so closely mimics the in vivo situation is useful.

A biologically active compound's toxicity and efficacy can be tested to characterize the biologically active compound. To test a biologically active compound, the formation of an accurate in vitro tissue model is highly desirable. A rotatable bioreactor is able to provide unique and useful in vitro conditions including an essentially quiescent three-dimensional culture in which cells may respond to biologically active compounds in a manner that closely represents the in vivo microenvironment.

The different classes of drugs have clearly different mechanisms of action but there are general features shared by the drug development process that the present invention addresses. For instance, in the case of anti-viral drugs, the complete life cycle of the viral particle offers opportunity for intervention. The viral life cycle at least includes initial transport of the viral particle and localization near the target cell, cell membrane penetration, genetic incorporation, viral sub-particle manufacturing, viral particle assembly, and viral release. These particular steps in the viral life cycle are steps at which biologically active compounds such as anti-viral drugs are directed and tested for efficacy. Therefore, such tests require the high fidelity cellular and multi-cellular tissue level behavior which closely mimic the in vivo microenvironment that are provided by expansion in a rotatable bioreactor, as in the present invention. Key examples of viruses which may preferably be tested by the method of the present invention include HIV, Bird Flu, Hepatitis, the herpes viruses, and in include the conventional DNA based as well as retroviral RNA (reverse transcriptase dependent) infectious viral particles. A similar program may be followed for preferably testing the effects of a biologically active compound preferably an anti-bacterial agent on a bacterial infection which may be tested for toxic syndromes with respect to toxin exposure and provide methods of corrective intervention (e.g. heavy metal exposure).

Other biologically active compounds may preferably be tested to determine their effects on normal cell and tissue functions and morphology, including whether the biologically active compounds can adjust these normal tissue and cell functions to potentially restore normal cell and tissue function or to inhibit diseased states. Without being bound by theory, in diseased states normal cell and tissue functions are over or under expressed, often due to regulatory and feedback mechanism problems. For instance, in the case of Adult Onset Diabetes, the cellular response to insulin (to admit glucose) is inadequate due to either rarified cell membrane insulin receptors, ineffective receptors, or blocked receptors (antibodies). The steady flow of drugs being tested to address Adult Onset Diabetes are directed to restoring these cellular functions and structures to normalcy. An in-vitro model which substantially mimics the in vivo situation, wherein cells having substantially the same three-dimensional geometry and cell-to-cell support and geometry as the in vivo microenvironment, and which sustains cellular and tissue functions that mimic the in vivo situation, is provided in the present invention for testing the effects of biologically active compounds, and therefore, characterize the same.

Preferably, at the same time that efficacy of a biologically active compound is being tested, the toxicity to the target cells and/or tissue as well as to other unrelated tissues, which may also be exposed to the drug and which may also be cultured to enhanced fidelity, in-vitro, can also be tested by the methods of the present invention. Preferably, abnormally functioning tissue and cells may be similarly cultured in a three-dimensional culture of a rotatable bioreactor for evaluation of the potential biologically active compound efficacy against the pathogenic target such as, but not limited to, malignantly transformed (cancerous) tissue. Some preferred biologically active compounds that may be tested against malignant tissue include, but are not limited to, chemotherapeutic, radiotherapeutic, anti-metastatic, tumor vasculature deprival, and nano-particle agents. Also, preferably, hybrid cell lines may be tested by the methods of the present invention.

It is notable that the future holds high promise for nano-particle (by the term “nano-particles” it is meant artificial bioactive particles) treatment modalities (for cancer as well as non-transformed disease state correction) and development of these will be dependent on accurate tissue culture in vitro models (both diseased and normal). Preferably, therefore, biologically active compounds that are directed to testing the nano-particle's functions including, but not limited to, the following functions homing, target identification, adherence, admittance, direct particle intervention, secondary particle functions such as drug release, particle lifetime/cycle, breakdown, and clearance can be tested by the present invention's expansion process in a rotatable bioreactor. Preferably, hybrids which consist of altered virus to meet therapeutic goals may be similarly tested.

The present invention also provides a method of preferably testing the mechanisms of pharmacologically modulating, altering, or correcting stem cell renewal and differentiative development along directed pathways to both renew the stem cell (or progenitor cell) pool and to produce the desired tissue (or committed progenitor lineage) from stem cells. The present invention, in a preferred embodiment, provides a method for accurately expanding stem cells in vitro while at the same time maintaining substantially the same three-dimensional geometry, cell-to-cell support and geometry as found in vivo. In such a preferred embodiment a three-dimensional culture having stem cells that can be tested for their response to soluble and direct contact control mechanisms, can also be preferably be tested for non direct clinical use such as engraftment quality assessment. To illuminate the broad applicability of the present invention, some additional preferred embodiments include testing diuretic performance and renal-toxicity on kidney tubule and matrix complexes; responses to blood pressure control treatments in smooth muscle expanded in a rotatable bioreactor; and testing biologically active compounds on autoimmune models.

The present invention is well adapted to carry out the objects and obtain the ends and advantages mentioned herein, as well as those inherent therein. Without departing from the scope of the invention, it is intended that all matter contained herein be interpreted as illustrative and not limiting. It will be apparent to those skilled in the art that various changes may be made in the invention without departing from the spirit and scope thereof and therefore the invention is not limited by that which is enclosed in the drawings and specifications, but only as indicated in the appended claims.

### Operative Method

In operation, a rotatable bioreactor preferably having a culture chamber of from 15 ml to about 2 L, is completely filled with the appropriate culture medium, preferably supplemented with albumin (5%) and also preferably G-CSF for human cells to be expanded, with room only for any intended additional volumes of culture medium, cells, biologically active compounds, and/or other preferred components of the culture medium of the intended three-dimensional culture. Preferably a controlled environment incubator completely surrounds the rotatable bioreactor and is preferably set for about 5% CO₂ and about 21% oxygen, and the temperature is preferably of from about 26° C. to about 41° C., and more preferably about 37° C.±2° C. Preferably the rotatable bioreactor may also have an integral thermometer, heater, and air control (including control of CO₂, O₂, and/or Nitrogen).

Initially, a stabilized culture environment is created in the culture medium. The rotation may preferably begin at about 10 RPM. 10 RPM is the preferred rate that produces a microcarrier bead orbital trajectory in which the beads do not accumulate appreciably at the chamber walls either by gravitational induced settling or by rotationally induced centrifugation. In this way, the rotatable bioreactor produces the minimal fluid velocity gradients and fluid shear stresses in the three-dimensional culture.

If cell attachment substrates are to be used, cell attachment substrates are preferably introduced either simultaneously or sequentially with cells into the culture chamber to give an appropriate density, preferably 5 mg of cell attachment substrate per ml of culture medium, and preferably the cell attachment substrate for the anchorage dependent cells are microcarrier beads. The cell mixture is preferably injected into the stabilized culture environment to initiate a three dimensional culture through an aperture in the culture chamber, preferably over a short period of time, preferably 2 minutes, so as to minimize cell damage while passing through the delivery system. Preferably, the cell mixture and/or the cell attachment substrate, if used, is delivered via a syringe.

After injection of the cells is complete, the culture chamber is quickly returned to initial rotation about a substantially horizontal axis, preferably in less than one (1) minute, preferably 10 RPM, thereby returning the fluid shear stress to the minimal level obtainable for the cells. During the initial loading and attachment phase, the cells are allowed to equilibrate for a short period of time, preferably of from 2 hours to 4 hours, more preferably for a time sufficient for transient flows to dampen out.

The biologically active compound to be tested in the present invention is introduced to the three-dimensional culture before, during, and/or after expansion of the cells. The method of introducing the biologically active compound will depend on the form that the biologically active compound takes (i.e. gas, liquid, or solid), and also the aperture through with the biologically active compound is to be introduced to the culture chamber. Furthermore, the concentration will also depend on the preferred test to ultimately be performed and the desired result of the biologically active compound.

As the expansion of the three-dimensional culture progresses it is expected that the size and sedimentation rate of the assembled cells increases, depending on the effect of a biologically active compound, and the system rotational rates may be increased (increasing in increments preferably of from about 1 to 2 RPM) in order to reduce the gravitationally induced orbital distortion from the ideal circular streamlines of the now increased diameter tissue pieces. Depending on the effects of the biologically active compound, the assembled cells, or cell mass, may increase or decrease in size. Either way, the rotation speed of the three-dimensional culture may need to be adjusted to prevent collision with the interior portion of the rotatable bioreactor. Wall impacts are not preferred, however, they are possible. A rotatable bioreactor, however, provides for any impact, if at all, to be of sufficiently low energetic impact so that it does not disrupt the quiescent three-dimensional culture.

During expansion, the rotational speed of the three-dimensional culture in the culture chamber may be assessed and adjusted so that the cells in the three-dimensional culture remain substantially at or about the horizontal axis. Increasing the rotational speed is warranted to prevent excessive wall impact, which is detrimental to further three-dimensional growth of delicate structure. For instance, an increase in the rotation is preferred if the cells in the three-dimensional culture fall excessively inward and downward on the downward side of the rotation cycle and excessively outward and insufficiently upward on the upward side of the rotation cycle. Optimally, the user is advised to preferably select a rotational rate that fosters minimal wall collision frequency and intensity so as to maintain the three-dimensional geometry and cell-to-cell support and cell-to-cell geometry of the cells. The preferred speed of the present invention is of from about 2 to about 30 RPM, and more preferably from about 10 to about 30 RPM.

The three-dimensional culture may preferably be visually assessed through the preferably transparent culture chamber and manually adjusted. The assessment and adjustment of the three-dimensional culture may also be automated by a sensor (for instance, a laser), which monitors the location of the cells within the culture chamber. A sensor reading indicating too much cell movement will automatically cause a mechanism to adjust the rotational speed accordingly.

After the initial loading of the cell mixture and preferably the attachment phase if cell attachment substrates are utilized (2 to 4 hours), in a preferred embodiment of the present invention, the TVEMF source is turned on and adjusted so that the TVEMF output generates the desired electromagnetic field in the three-dimensional culture in the culture chamber. The TVEMF may also preferably be applied to the three-dimensional culture during the initial loading and attachment phase. It is preferable that TVEMF is supplied to the three-dimensional culture for the length of the expansion time until it is terminated.

The size of the electrically conductive coil, and number of times it is wound around the culture chamber of the rotatable TVEMF bioreactor, are such that when a TVEMF is supplied to the electrically conductive coil a TVEMF is generated within the three-dimensional culture in the culture chamber of the rotatable TVEMF bioreactor. The TVEMF is preferably selected from one of the following: (1) a TVEMF with a force amplitude less than 100 gauss and slew rate greater than 1000 gauss per second, (2) a TVEMF with a low force amplitude bipolar square wave at a frequency less than 100 Hz., (3) a TVEMF with a low force amplitude square wave with less than 100% duty cycle, (4) a TVEMF with slew rates greater than 1000 gauss per second for duration pulses less than 1 ms., (5) a TVEMF with slew rate bipolar delta function-like pulses with a duty cycle less than 1%, (6) a TVEMF with a force amplitude less than 100 gauss peak-to-peak and slew rate bipolar delta function-like pulses and where the duty cycle is less than 1%, (7) a TVEMF applied using a solenoid coil to create uniform force strength throughout the cell mixture, (8) and a TVEMF applied utilizing a flux concentrator to provide spatial gradients of magnetic flux and magnetic flux focusing within the cell mixture. The range of frequency in oscillating electromagnetic force strength is a parameter that may be selected for achieving the desired stimulation of the cells in the three-dimensional culture. However, these parameters are not meant to be limiting to the TVEMF of the present invention, and as such may vary based on other aspects of this invention. TVEMF may be measured for instance by standard equipment such as an EN131 Cell Sensor Gauss Meter.

The rapid cell expansion and increasing total metabolic demand may necessitate intermittent addition of preferable components enriching the culture medium in the three-dimensional culture including, but not limited to, nutrients, fresh growth medium, growth factors, hormones, and cytokines. This addition is preferably increased as necessary to maintain glucose and other nutrient levels. During the rapid cell and tissue expansion in the rotatable bioreactor, culture medium comprising waste nay preferably be removed as necessary. Samples of the biological component may also be removed from the three-dimensional culture to be tested and the culture chamber rotation may be temporarily stopped to allow practical handling. The three-dimensional culture may preferably be allowed to progress beyond the point at which it is possible to select excellent cells orbits; at a point when gravity has introduced constraints which somewhat degrade performance in terms of a low shear three-dimensional culture. Furthermore, after expansion, the cells may be used for therapeutic purposes including for the regeneration of tissue, research, and treatment of disease.

The following examples are preferred illustrations of the invention, but they are not intended to limit the invention thereto.

### EXAMPLE 1

### Expansion of Adult Stem Cells and a Biologically Active Compound

Preparation

A 75 ml culture chamber of a rotatable bioreactor, illustrated in the preferred embodiment of FIGS. 1 and 2, may preferably be prepared by washing with detergent and germicidal disinfectant solution (Roccal II) at the recommended concentration for disinfection and cleaning followed by copious rinsing and soaking with high quality deionized water. The rotatable bioreactor may be sterilized by autoclaving then rinsed once with culture medium. If a disposable culture chamber of a rotatable bioreactor is utilized then preferably the disposable culture chamber is already sterilized and merely needs to be removed from any packaging and assembled onto the motor. For the preferred embodiment having an electrically conductive coil, the electrically conductive coil is connected to the TVEMF source of the rotatable bioreactor.

Expansion of Peripheral Blood Stem Cells

The rotatable bioreactor may preferably be filled with culture medium consisting of Isocove's modified Dulbecco's medium (IMDM) (GIBCO, Grand Island N.Y.), supplemented with 5% humnani albumin, 100 ng/ml recombinanit human C-CSF (Amgen Inc., Thousand Oaks, Calif.), and 100 ng/ml recombinant human stem cell factor (SCF) (Amgen). In addition, D-Penicillamine [D(−)-2-Amino-3-mercapto-3-methylbutonoic acid] (Signa-Aldrich) a copper chelating agent, dissolved in DMSO, may preferably be introduced to the culture medium in the rotatable bioreactor in an amount of 10 ppm. Adult stem cells from peripheral blood (CD34+/CD38−) may preferably be placed in the culture chamber of the rotatable bioreactor at a concentration of 0.75×10⁶ cells/ml. Preferably, the culture chamber is equilibrated before the cell mixture is placed therein. If a culture medium flow loop is utilized, as depicted in the preferred embodiment in FIG. 3, then equilibration of the culture medium is also preferable to create a stabilized culture environment. The stabilized culture environment provides for substantially low stress shear levels for the addition of the cell mixture.

The motor should be turned on, preferably to a rate of approximately 30 RPM. If the culture chamber and culture medium therein have been equilibrated the speed of rotation should be slowly returned to the preferred rate of rotation. The rotation of the rotatable bioreactor may preferably be assessed every day, and adjusted to maintain the rotation at a speed to prevent wall impact and keep the cells of the three-dimensional culture substantially in the middle of the culture chamber. The TV EMF source may also preferably be turned on to the preferred gauss and oscillating range, preferably from about 1 mA to about 1,000 mA. The expansion should preferably be allowed to proceed for seven days and was then terminated, at which time, the cells were tested.

Samples and Results

At least two samples of peripheral blood stem cells should preferably be expanded in the rotatable bioreactor under the conditions above stated. Sample 1 should be expanded for seven days and then the viability assessed under a microscope. It is expected that the cells in the first sample will remain healthy and multiply. A biologically active compound should preferably be introduced to Sample 2 at the initiation of the three-dimensional culture. The biologically active compound may preferably be 3 ppm Pseudomonas aeruginosa bacteria. The expansion conditions between Samples 1 and 2, other than the bacterial should preferably be the same. After seven days, the experimental cultures should be terminated and the viability of the cells assessed under a microscope. It is expected that microscopic examination will reveal that the cells from Sample 2, containing a bacterial biologically active compound, will be dead while the cells in Sample 1 will remain viable and healthy. Such results predict that this preferred bacteria is likely to be toxic if allowed to enter the peripheral blood stream. The viability of the cells may be determined by any known and accepted method known in the art.

### EXAMPLE 2

### Expansion of Rat Renal Cells and a Biologically Active Compound

Preparation

The rotatable bioreactor should be prepared as in Example 1 above.

Expansion of Rat Renal Cells

Rat renal cells may preferably be isolated from renal cortex harvested from euthenized Sprague Dawley rats (Harlan Sprague-Dawley, Cleveland Ohio). In brief, renal cortex may preferably be dissected out with scissors, minced finely in a renal cell buffer 137 mmol NaCl, 5.4 mmol KCl, 2.8 mmol CaCl2, 1.2 mmol MgCl2, 10 mmol HEPES-Tris, pH 7.4. The minced tissue may preferably be placed in 10 ml of a solution of 0.1% Type IV collagenase and 0.1% trypsin in normal saline. The solution containing the tissue may preferably be incubated in a 37° C. shaking water bath for 45 minutes with intermittent titration. The cells may preferably be place in a centrifuge and centrifuged gently (800 rpm for 5 minutes), the supernatant aspirated, the cells resuspended in 5 ml renal cell buffer with 0.1% bovine serum, and passed through a fine (70 mm) mesh. The fraction passing through the mesh may preferably be layered over a discontinuous gradient of 5% bovine serum albumin and centrifuged gently (800 rpm for 5 minutes). The supernatant should again be discarded leaving a cell pellet of rat renal cells. At this point, the cells may preferably be frozen (preferably at −80° C., more preferrably in liquid nitrogen) as needed for future use.

The rat renal cell pellet may preferably be resuspended in DMEM/F-12 medium (ciprofloxacin and fungizone treated), in a concentration that is approximately 1×10⁶ cell/ml. At least two samples of cells (1×10⁶ cell/ml) are preferably expanded in the culture chamber of a rotatable bioreactor. The rotatable bioreactor should be placed in a 5% CO₂ 95% O₂ incubator, or have an integral air and temperature gage adjusted thereto. The rat renal cells should preferably be expanded for 7 days.

Samples and Results

Sample 1 of the rat renal cells should be expanded without any additives including any biologically active compounds. A biologically active compound, 10 ppm of diisooctyl phthalate plasticizer, should be introduced to Sample 2 preferably at the initiation of the three-dimensional culture. Both Samples, 1 and 2, should have the viability of the cells assessed, preferably after 7 days, by methods known in the art such by microscopic determination. It is expected that the cells in Sample 1 will expand to at least seven times as many as were placed into the rotatable bioreactor. On the other hand, it is expected that the majority of the cells in Sample 2 will die. Such results predict that diisooctyl phthalate is toxic if allowed to be introduced into the body and to accumulate in the renal cells. Other than the biologically active compound, all other conditions are preferably the same as between Samples 1 and 2. In addition, the culture conditions and the rotation of the rotatable bioreactor should preferably be the same as in Example 1. However, the three-dimensional culture is preferably not exposed to a TV EMF in this Example 2.

### EXAMPLE 3

### Expansion of Rat Renal Cells and a Biologically Active Compound

Preparation

The rotatable bioreactor should be prepared as in Example 1 above.

Expansion of Rat Renal Cells, Samples and Results

In Example 3, Example 2 should be repeated except that in the Sample 2, the diisooctyl phthalate plasticizer is preferably replaced with 10 ppm Cisplatinium. The test is preferably repeated 10 times under the same conditions as in Example 2. In almost all instances, it is expected that the cells in Sample 1 will remain viable. It is expected that the cells in Sample 2, and in the majority of cases having 10 ppm Cisplatinum, the rat renal cells will remain healthy and viable. Such results predict, therefore, that adding 10 ppm Cisplatinum to rat renal cells and expanding them in a rotatable bioreactor produces no adverse effects, ultimately suggesting that Cisplatinum may, in fact, prove helpful in preventing renal failure. Additional studies should be conducted on prevention of renal failure by using Cisplatinum before using Cisplatinum on humans.

### EXAMPLE 4

### Expansion of Peripheral Blood Stem Cells and a Biologically Active Compound

Preparation

The rotatable bioreactor should be prepared as in Example 1 above.

Expansion of Peripheral Blood Stem Cells

The rotatable bioreactor may preferably be filled with culture medium consisting of Isocove's modified Dulbecco's medium (IMDM) (GIBCO, Grand Island N.Y.), supplemented with 5% human albumin, 100 ng/ml recombinant human G-CSF (Amgen Inc., Thousand Oaks, Calif.), and 100 ng/ml recombinant human stem cell factor (SCF) (Amgen). In addition, D-Penicillamine [D(−)-2-Amino-3-mercapto-3-methylbutonoic acid] (Sigma-Aldrich) a copper chelating agent, dissolved in DMSO, may preferably be introduced to the culture medium in the rotatable bioreactor in an amount of 10 ppm. Adult stem cells from peripheral blood (CD34+/CD38−) may preferably be placed in the culture chamber of the rotatable bioreactor at a concentration of 0.75×10⁶ cells/ml.

Two samples of peripheral blood stem cells should preferably be prepared by this method. Sample 1 should preferably be from an individual with no known liver damage. Sample 2 should preferably be from an individual with known liver damage. The samples should be prepared as above and placed in two different rotatable bioreactors under the conditions noted in Example 1 and at the same concentrations. The biologically active compound, 20 ppm of acetaminophen, should be added to Sample 2 at the initiation of the three-dimensional culture. Both Samples 1 and 2 should preferably be exposed to a TVEMF of from about 1 mA to about 1,000 mA as in Example 1 for the duration of the expansion process.

Results

Preferably, at the end of 14 days each sample's viability should be assessed and the number of cells counted, for example under a microscope with a hematocytometer. It is expected that the cells in Sample 1 expand to at least ten times the number that were placed in the rotatable bioreactor. It is also expected that the cells in Sample 2 neither die nor grow, but rather, remain unchanged. Such results predict a potential problem of regenerating liver tissue in the presence of 20 ppm acetaminophen. More testing should be performed to determine the effects of exposing liver cells to acetaminophen.

It is expected, therefore, that rapid and significant cell expansion is accomplished by expansion in the rotatable bioreactor of the present invention, as described herein. It is also expected that the rapid and significant expansion is accompanied by a three-dimensionality and cell-to-cell interactions that is substantially similar to the in vivo microenvironment.

Various changes may be made in the invention without departing from the spirit and scope thereof, and therefore, the invention is not limited by that which is enclosed in the drawings and specification, including the examples.

I claim:

1. A method of characterizing a biologically active compound comprising:
placing a cell mixture into a rotatable bioreactor to initiate a three-dimensional culture wherein the three-dimensional culture comprises cells and a biological component;
controllably expanding the cells in the three-dimensional culture while at the same time maintaining the cells three dimensional geometry and cell-to-cell support and geometry by rotating the rotatable bioreactor;
introducing a biologically active compound to the three dimensional culture; and
testing the biological component using a test to characterize the pharmaceutical compound.

2. The method as in claim 1 wherein the biological component is selected from the group consisting of a cell, a portion of a cell, secreted materials (mucin, collagen, matrix), secreted hormones, secreted intercellular structural components, introduced structural matrices, adherence matrices, growth substrates, nanoparticles, intercellular soluble signals, cell membrane surface markers, membrane bound enzymes, immune identity markers, adherence molecules, vacuoles, stored and released neurotransmitters, cellular internal specialized machinery, glycogen, culture media, compounds under test, suspected toxins under test, reagents under test, fungus, a conjugated complexes, tissue, enzymes, DNA, RNA, virus, protein, artificial bioactive particles, and a gene.

3. The method as in claim 2 wherein the rotatable bioreactor comprises a rotating culture chamber wall and wherein a portion of the three-dimensional culture is fixed with respect to the rotating culture chamber wall.

4. The method as in claim 1 wherein the step of controllably expanding the cells further comprises exposing the cells to a time varying electromagnetic force.

5. The method as in claim 1 wherein the cells are expanded to at least seven times the number that were placed in the rotatable bioreactor.

6. The method as in claim 1 wherein the cells are selected from the group consisting of eukaryote, prokaryote, animal, fungus, plant, abnormally functioning cells, nano-particle containing cells, hybrid cells, altered virus containing cell hybrids.

7. The method as in claim 2 wherein the cells are selected from the group consisting of eukaryote, prokaryote, animal, fungus, plant, abnormally functioning cells, nano-particle containing cells, hybrid cells, altered virus containing cell hybrids.

8. The method as in claim 4 wherein the cells are selected from the group consisting of eukaryote, prokaryote, animal, fungus, plant, abnormally functioning cells, nano-particle containing cells, and hybrid cells, altered virus containing cell hybrids.

9. The method as in claim 6 wherein the animal cells are mammalian adult stem cells.

10. The method as in claim 7 wherein the animal cells are mammalian adult stem cells.

11. The method as in Claim 8 wherein the animal cells are mammalian adult stem cells.

12. The method as in claim 1 wherein the test is for at least one of the group consisting of engraftment quality, toxicity, efficacy, pathology, tumorogenicity, genetic expression, karyotype, growth rate characteristics, multi-cellular morphology, individual cellular morphology, inter-cellular relationships, metabolic measures, a portion of a viral life cycle, diuretic performance, renal-toxicity, blood pressure control, and nano-particle functions.

13. The method as in claim 1 wherein the biologically active compound is in a form selected from a group consisting of powder, liquid, vapor, and gas.

14. The method as in claim 1 wherein the biologically active compound is at least one selected from the group consisting of a protein, at least one cell, a toxin, a reagent, a chemical, a gas, a metal, a composite of metals, radiation, at least one nano-particle, at least one virus, a protein, anti-bacterial, electroporation, chemical poration, an activated derivative of an immune cell, and water.

15. The method as in claim 9 wherein the test is for characterizing at least one selected from the group consisting of the mechanisms of pharmacologically modulating stem cell renewal, altering stem cell renewal, correcting stem cell renewal, pharmacologically modulating stem cell differentiation, altering stem cell differentiation, and correcting stem cell differentiation.

16. The method as in claim 10 wherein the test is for characterizing at least one selected from the group consisting of the mechanisms of pharmacologically modulating stein cell renewal, altering stem cell renewal, correcting stem cell renewal, pharmacologically modulating stem cell differentiation, altering stem cell differentiation, and correcting stem cell differentiation.

17. The method as in claim 11 wherein the test is for characterizing at least one selected from the group consisting of pharmacologically modulating stem cell renewal, altering stem cell renewal, correcting stem cell renewal, pharmacologically modulating stem cell differentiation, altering stem cell differentiation, and correcting stem cell differentiation,

18. The method as in claim 1 wherein the rotatable bioreactor is rotated at a rate of about 1 revolutions per minute to about 120 revolutions per minute.

19. The method as in claim 1 wherein the rotatable bioreactor is rotated as a rate of about 2 revolutions per minute to about 30 revolutions per minute.

20. The method as in claim 1 wherein the rotatable bioreactor is rotated as a rate of about 10 revolutions per minute to about 30 revolutions per minute.

21. The method as in claim 1 wherein the biologically active compound is introduced before the step of controllably expanding the cells.

22. The method as in claim 1 wherein the biologically active compound is introduced during the step of controllably expanding the cells.

23. The method as in claim 1 wherein the biological component is tested before placing the cell mixture into the rotatable bioreactor.

24. The method as in claim 1 wherein the biological component is tested during the step of controllably expanding the cells.

25. The method as in claim 1 wherein the biological component is tested after the step of controllably expanding the cells.

26. The method as in claim 1 wherein the biological component is tested during and after the step of controllably expanding the cells.

27. The method as in claim 1 wherein the biological component is tested before, during, and after the step of controllably expanding the cells.

28. The method as in claim 1 further comprising using the biological component for mammalian tissue engraftment.
